(19) United States
(12) Patent Application Publication
(10) Pub. No.: US 2009/0248130 A1
(43) Pub. Date: Oct. 1, 2009

(54) NITINOL ALLOY DESIGN AND COMPOSITION FOR VASCULAR STENTS

(75) Inventor: John F. Boylan, Murrieta, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Cardiovascular Systems, Inc., Santa Clara, CA (US)

(21) Appl. No.: 12/369,360

(22) Filed: Feb. 11, 2009

Related U.S. Application Data
(63) Continuation of application No. 09/452,516, filed on Dec. 1, 1999.

(51) Int. Cl. A61F 2/06

(52) U.S. Cl. 623/1.11; 623/1.23; 623/1.15

(57) ABSTRACT

A stent and a delivery system for implanting the stent in a body lumen is disclosed. The stent is made from a superelastic alloy such as nickel-titanium or nitinol, and includes a ternary element in order to minimize the stress hysteresis of the superelastic material. The stress hysteresis is defined by the difference between the loading plateau stress and the unloading plateau stress of the superelastic material. The resulting delivery system has a small profile and includes a sheath covering the stent that has a thin wall.

### BACKGROUND OF THE INVENTION

The present invention generally relates to self-expanding endoprosthesis devices, in particular self-expanding intraluminal vascular grafts, generally called stents, adapted to be implanted in a body lumen, such as carotid arteries, coronary arteries, peripheral arteries, veins, or other vessels to maintain the patency of the lumen. These devices are frequently used in the treatment of atherosclerotic stenosis in blood vessels especially after percutaneous transluminal angioplasty (PTA) or percutaneous transluminal coronary angioplasty (PTCA) procedures, with the intent to reduce the likelihood of restenosis of a vessel. Stents are also used to support a body lumen, tack-up a flap or dissection in a vessel, or in general where the lumen is weak to add support. The present invention also relates to an intraluminal vascular graft that can be used in essentially any body lumen.

In expandable stents that are delivered with expandable catheters, such as balloon catheters, the stents are positioned over the balloon portion of the catheter and are expanded from a reduced diameter to an enlarged diameter greater than or equal to the inner diameter of the arterial wall, by inflating the balloon. Stents of this type can be expanded to an enlarged diameter by deforming the stent, by engagement of the stent walls with respect to one another, and by one way engagement of the stent walls together with endothelial growth onto and over the stent. Other stents are self-expanding, through the properties of the material constituting the stent or by design. Examples of intravascular stents can be found in U.S. Pat. No. 5,292,331 (Boneau); U.S. Pat. No. 4,580,568 (Gianturco); U.S. Pat. No. 4,856,516 (Hillstead); U.S. Pat. No. 5,092,877 (Pinchuk); and U.S. Pat. No. 5,514,154 (Lau et al.), which are incorporated herein by reference in their entirety.

The problems with some prior art stents, especially those of the expandable type, is that they are often stiff and inflexible. Often, the expandable type stents are formed from stainless steel alloys and the stents are constructed so that they are expanded beyond their elastic limit. Such stents are permanently deformed beyond their elastic limits and are capable of holding open a body lumen and maintaining patency of the body lumen. There are several commercially available stents that are widely used and generally implanted in the coronary arteries after a PTCA procedure.

One class of stents is implanted in vessels that are closer to the surface of the body, such as in the carotid arteries in the neck or in peripheral arteries and veins in the leg. Because these stents are so close to the surface of the body they are particularly vulnerable to impact forces that can partially or completely collapse the stent and thereby block fluid flow in the vessel. Since the prior art stents are plastically deformed, once collapsed or crushed they will remain so, permanently blocking the vessel. Thus, the prior art stents can pose an undesirable condition to the patient.

Other forces can impact the prior art stents and cause similar partial or total vessel blockage. Under certain conditions, muscle contractions might cause the prior art stents to partially or totally collapse and restrict blood flow in the vessel in which they are implanted.

Such important applications as mentioned above have prompted stent designers to use superelastic or shape memory alloys in their stent to exploit the materials' properties. An example of such shape memory alloy stents is disclosed in, for example, European Patent Application Publication No. EP0873734A2, entitled “Shape Memory Alloy Stent.” This publication suggests a stent for use in a lumen in a human or animal body having a generally tubular body formed from a shape memory alloy which has been treated so that it exhibits enhanced elastic properties. In particular, in the stress-strain curve exhibiting loading and unloading of the shape memory alloy material, the applicant suggests using a composition that results in a large difference between the loading and unloading curves, otherwise known as a wide hysteresis. The wide hysteresis means that the inward force required to compress the stent transversely once in place in the lumen is relatively high, while the outward force that the stent exerts on the lumen as it attempts to revert to its original undeformed configuration is relatively low. This can mean that the lumen will be resistant to being crushed by externally applied forces which can be a problem in the case of lumens close to the surface such as arteries in the thigh and neck. The publication further suggests use of specified ternary elements in a nickel titanium alloy to obtain a stent exhibiting a wider hysteresis in the stress-strain behavior in a loading and unloading cycle.

The evolution of superelastic and shape memory alloy stents progressed to use of ternary elements in combination with nickel-titanium alloys to obtain specific material properties. Use of a ternary element in a superelastic stent is shown in, for example, U.S. Pat. No. 5,907,893 to Zadno-Azizi et al. As a general proposition, there have been attempts at adding a ternary element to nickel-titanium alloys as disclosed in, for instance, U.S. Pat. No. 5,885,381 to Mitose et al.

On the other hand, the conventional efforts of using a ternary element in a superelastic material for a stent have focused only on a wider hysteresis in the stress-strain behavior in a loading or unloading cycle of the stent. Unfortunately, the greater the difference between the loading and unloading stress plateaus, the stronger the delivery system must be to accommodate any given level of stent performance. Typically, a stronger delivery system must also be larger and bulkier. This is a major drawback to conventional superelastic stents and delivery systems when the stent must be delivered through tortuous vessels at remote locations in the human anatomy.

What has been needed and heretofore unavailable in the prior art is a superelastic stent and delivery system that applies a ternary element to the superelastic alloy in order to minimize the hysteresis. That hysteresis is defined by the difference between the loading and unloading plateau stresses of the material as plotted on a stress-strain curve. The present invention satisfies these needs.

### SUMMARY OF THE INVENTION

The present invention is directed to a stent and a delivery system for implanting the stent in a body lumen, comprising a cylindrically-shaped stent including a superelastic alloy, wherein the alloy includes a ternary element, and wherein the alloy further includes a substantially small stress hysteresis; and a delivery system including a sheath having a distal end and a proximal end, wherein the stent is disposed inside the sheath at the distal end, and wherein the sheath has a small profile.

In a preferred embodiment, the superelastic alloy includes binary nickel-titanium alloys that exhibit superelasticity and have an unusual stress-strain relationship. More precisely, the superelastic curve is characterized by regions of nearly constant stress upon loading (referred to as the loading plateau stress) and unloading (unloading plateau stress). The loading plateau stress is always larger than the unloading plateau stress. The loading plateau represents the period during which martensite is being stress-induced in favor of the original austenitic structure. As the load is removed, the stress-induced martensite transforms back into austenite along the unloading plateau.

Self-expanding nitinol stents are collapsed (that is, loaded) and then constrained within a delivery system. At the point of delivery, the stent is released (that is, unloaded) and allowed to return to its original diameter. The stent is designed to perform various mechanical functions within the lumen, all of which are based upon the lower unloading plateau stress.

Importantly, the higher loading plateau stress therefore establishes the mechanical resistance the stent exerts against the delivery system. The greater the difference between these two plateaus is, the wider the hysteresis curve, and the stronger the delivery system must be to accommodate any given level of stent performance. The greater difference is described as a wide hysteresis. The conventional superelastic stent with a ternary element is designed to have a wider hysteresis resulting in a larger profile delivery system.

In the preferred embodiment of the present invention, however, an object is to decrease the stress hysteresis defined by the loading and unloading stress plateaus. This is accomplished by using a ternary element in addition to the superelastic alloy. As a result, the present invention stent and delivery system will enjoy an overall reduced delivery system profile for any given level of stent mechanical performance. Moreover, because of the smaller hysteresis and lower loading plateau stress for a given level of performance, the delivery system including the sheath can be made of a thinner wall material, leading to better flexibility.

As mentioned above, a preferred superelastic alloy is nickel-titanium or nitinol. In the exemplary embodiment, the ternary element may be palladium, chromium, iron, cobalt, vanadium, manganese, boron, copper, aluminum, tungsten, or zirconium.

Other features and advantages of the present invention will become more apparent from the following detailed description of the invention when taken in conjunction with the accompanying exemplary drawings.

### BRIEF DESCRIPTION OF THE DRAWINGS

FIG. 1 is a partial cross-sectional view of a stent delivery system.

FIG. 2 shows, in a cross-sectional view, the stent delivery system of FIG. 1 with an optional expandable balloon.

FIG. 3 is a side elevational view, partially in section, depicting a stent mounted on a delivery catheter and expanded within a damaged vessel, pressing a damaged vessel lining against the vessel wall.

FIG. 4 is a side elevational view, partially in section, depicting an expanded stent within the vessel after withdrawal of the delivery catheter.

FIG. 5 is a plan view of the flattened strut pattern of an exemplary embodiment of a superelastic stent.

FIG. 6 is a typical stress-strain curve for a superelastic material.

### DETAILED DESCRIPTION OF THE PREFERRED EMBODIMENTS

During PTCA procedures it is common to use a dilation catheter to expand a diseased area to open the patient's lumen so that blood freely flows. Despite the beneficial aspects of PTCA procedures and its widespread and accepted use, it has several drawbacks, including the possible development of restenosis and perhaps acute thrombosis and sub-acute closure. This recurrent stenosis has been estimated to occur in seventeen to fifty percent of patients despite the initial PTCA procedure being successful. Restenosis is a complex and not fully understood biological response to injury of a vessel which results in chronic hyperplasia of the neointima. This neonintimal hyperplasia is activated by growth factors which are released in response to injury. Acute thrombosis is also a result of vascular injury and requires systemic antithrombotic drugs and possibly thrombolytics as well. This therapy can increase bleeding complications at the catheter insertion site and may result in a longer hospital stay. Sub-acute closure is a result of thrombosis, elastic recoil, and/or vessel dissection.

Several procedures have been developed to combat restenosis and sub-acute or abrupt closure, one of which is the delivery and implanting of an intravascular stent. Stents are widely used throughout the United States and in Europe and other countries. Generally speaking, the stents can take numerous forms, however, most common is a generally cylindrical hollow tube that holds open the vascular wall at the area that has been dilated by a dilation catheter. One highly regarded stent used and sold in the United States is sold under the tradename ACS Multi-Link Stent, which is made by Advanced Cardiovascular Systems, Inc., Santa Clara, Calif.

The stents of the present invention can have virtually any configuration that is compatible with the body lumen in which they are implanted. The stent should be configured so that there is a substantial amount of open area and preferably the open area to metal ratio is at least 80%. The stent also should be configured so that dissections or flaps in the body lumen wall are covered and tacked up by the stent.

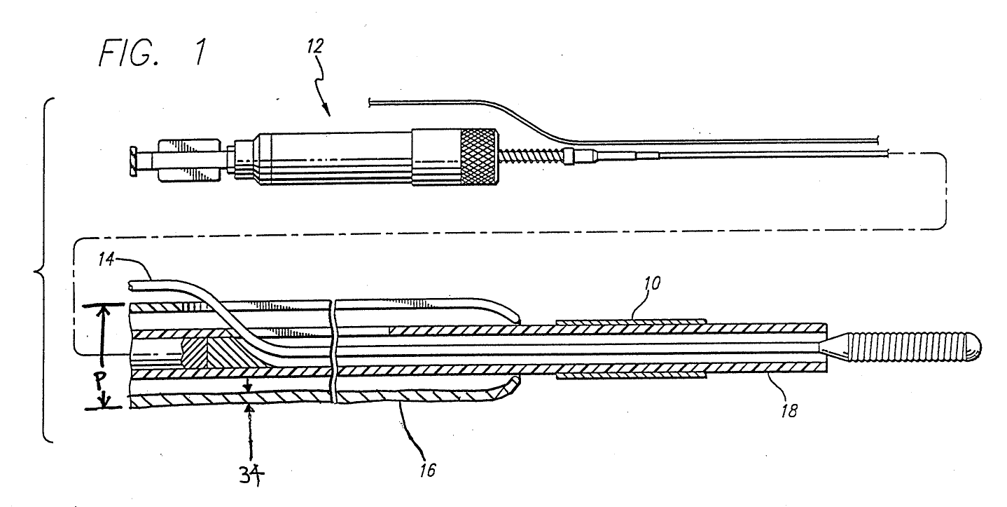

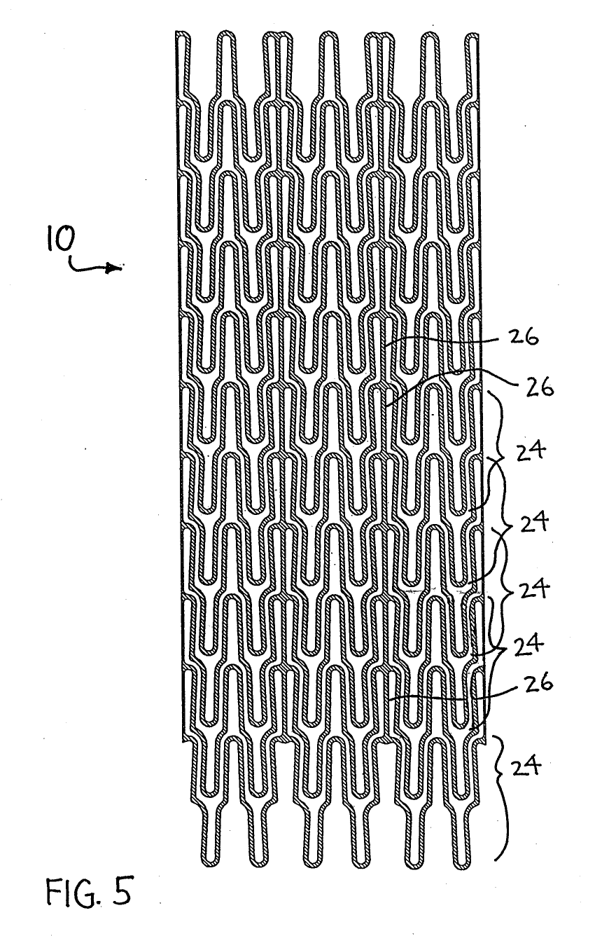

Referring to FIGS. 1 and 5, in a preferred embodiment, stent 10 of the present invention is formed partially or completely of alloys such as nitinol (NiTi) which have superelastic (SE) characteristics. Stent 10 is somewhat similar to the stent disclosed in U.S. Pat. No. 5,569,295, “Expandable Stents and Method for Making Same,” issued to Lam on Oct. 29, 1996, which is incorporated herein by reference in its entirety. Some differences of the present invention stent from that disclosed in the '295 patent is that the present invention stent is constructed of a superelastic material, and the strut pattern has changed. Of course, the configuration of stent 10 is just one example of many stent configurations that are contemplated by the present invention.

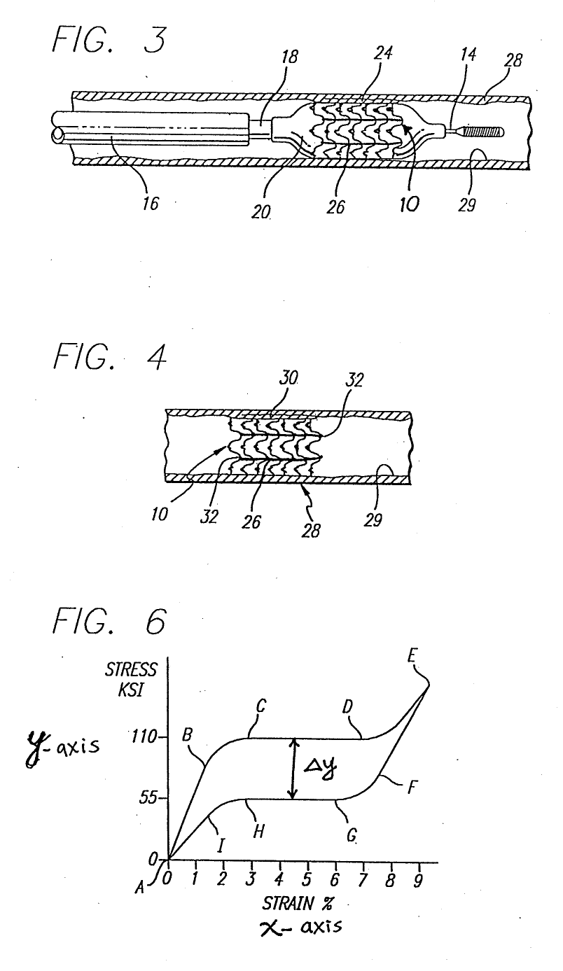

In keeping with the present invention, and turning to FIGS. 3, 4, and 5, stent 10 preferably includes a plurality of radially expandable cylindrical elements 24 disposed generally coaxially and interconnected by members 26 disposed between adjacent cylindrical elements 24. The shape of the struts are designed so they can preferably be “nested.” This is best seen from the flattened plan view of FIG. 5. The serpentine shaped struts are nested such that the extended portions of the struts of one cylindrical element 24 intrude into a complementary space within the circumference of an adjacent cylindrical element. In this manner, the plurality of cylindrical elements 24 can be more tightly packed lengthwise.

As introduced above, an exemplary stent of the present invention includes a superelastic material. The term “superelastic” refers to an isothermal transformation, more specifically stress inducing a martensitic from an austenitic phase. Alloys having superelastic properties generally have at least two phases: a martensitic phase, which has a relatively low tensile strength and which is stable at relatively low temperatures, and an austenitic phase, which has a relatively high tensile strength and which is stable at temperatures higher than the martensitic phase. Superelastic characteristics generally allow the metal stent to be deformed by collapsing and deforming the stent and creating stress which causes the NiTi to change to the martensitic phase. The stent is restrained in the deformed condition to facilitate the insertion into a patient's body, with such deformation causing the phase transformation. Once within the body lumen, the restraint on the stent is removed, thereby reducing the stress therein so that the superelastic stent can return to its original undeformed shape by the transformation back to the austenitic phase.

Returning to FIG. 1, the graphic illustrates, in a partial cross-sectional view, a rapid exchange stent delivery system that includes manipulating device 12, guidewire 14, delivery sheath 16, and intravascular catheter 18. This delivery system is just one example of a delivery system that may be used with the present invention. More details of this type of delivery system may be found in, for example, U.S. Pat. No. 5,458,615, “Stent Delivery System,” issued to Klemm et al. on Oct. 17, 1995, which is incorporated herein by reference in its entirety. Other delivery systems such as an over-the-wire delivery system may be used without departing from the scope of the instant invention.

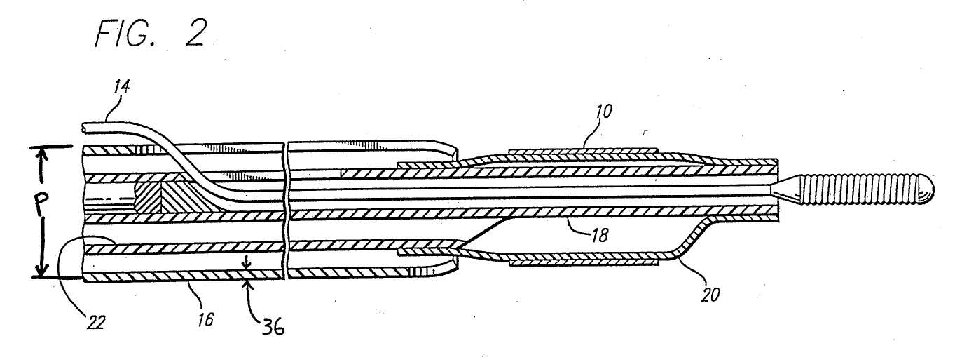

FIG. 2 depicts in a partial cross-sectional view a variation on the delivery system of FIG. 1, and includes optional expandable balloon 20 and optional balloon inflation lumen 22. Stent 10 is disposed over expandable balloon 20, and the entire assembly is kept underneath delivery sheath 16 until the moment stent 10 is deployed.

FIGS. 1 and 2 also depict delivery systems having a small delivery profile P. This reduced profile P is a beneficial attribute of the present invention stent and delivery system as a result of the stress-strain hysteresis curve of the superelastic material being minimized. This novel approach is described more fully below.

Stent 10 is preferably formed from a superelastic material such as NiTi and undergoes an isothermal transformation when stressed. The stent is first compressed to a delivery diameter, thereby creating stress in the NiTi alloy so that the NiTi is in a martensitic state having relatively low tensile strength. While still in the martensitic phase, the stent is mounted onto a catheter by known methods such as adhesives, or other restraining means. Alternatively, stent 10 can be mounted within delivery sheath 16 so that stent 10, which tends to spring back to a larger diameter, pushes radially outwardly against the inside diameter of sheath 16.

In its delivery diameter P, the overall diameter of the stent and catheter are less than the inside diameter of artery 28 or the vessel in which they are inserted. After stent 10 is inserted into the artery or other vessel, the stress exerted by stent 10 may be released by withdrawing delivery sheath 16 in a proximal direction, whereupon stent 10 immediately expands and returns to its original, undeformed shape by transforming back to the more stable austenitic phase. If expandable balloon 20 of FIG. 2 is implemented, stent 10 may be further expanded by inflation of expandable balloon 20 via balloon inflation lumen 22 by known methods.

FIG. 4 illustrates stent 10 in the expanded condition after the delivery system has been removed. If an external force is then applied to the artery, the stent temporarily at least partially collapses or deforms. As the stent deforms, stress in the NiTi alloy causes a phase transformation from the austenitic to the martensitic phase. When the external force is removed, the stress in stent 10 is removed so that the stent quickly transforms back from the martensitic phase to the austenitic phase. As this almost instantaneous transformation occurs, stent 10 returns to its fully expanded state and the artery remains open. When superelastic stent 10 is implanted in an artery, it maintains the patency of the artery while minimizing the risk of permanent arterial collapse at the implant site if the stent is temporarily deformed due to external forces. Thus, stent 10 imparts crush-resistant support at the implant site.

When stress is applied to a specimen of a metal such as nitinol exhibiting superelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase. As the phase transformation progresses, the alloy undergoes significant increases in strain with little or no corresponding increases in stress. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation.

If the load on the specimen is removed before any permanent deformation has occurred, the martensite specimen will elastically recover and transform back to the austenitic phase. The reduction in stress first causes a decrease in strain. As stress reduction reaches the level at which the martensitic phase transforms back into the austenitic phase, the stress level in the specimen will remain essentially constant (but less than the constant stress level at which the austenitic crystalline structure transforms to the martensitic crystalline structure until the transformation back to the austenitic phase is complete); i.e., there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as superelasticity.

The prior art makes reference to the use of metal alloys having superelastic characteristics in medical devices which are intended to be inserted or otherwise used within a patient's body. See, for example, U.S. Pat. No. 4,665,905 (Jervis) and U.S. Pat. No. 4,925,445 (Sakamoto et al.), which are incorporated by reference herein in their entirety.

FIG. 6 illustrates an example of a preferred stress-strain relationship of an alloy specimen, such as stent 10, having superelastic properties as would be exhibited upon tensile testing of the specimen. The relationship is plotted on x-y axes, with the x axis representing strain and the y axis representing stress. For ease of illustration, the x-y axes are labeled with typical pseudoelastic nitinol stress from 0 to 110 ksi and strain from 0 to 9 percent, respectively.

Looking at the plot itself in FIG. 6, the line from point A to point B represents the elastic deformation of the specimen. After point B the strain or deformation is no longer proportional to the applied stress and it is in the region between point B and point C that the stress-induced transformation of the austenitic phase to the martensitic phase begins to occur. There also can be an intermediate phase, called the rhombohedral phase, depending upon the composition of the alloy.

At point C moving toward point D, the material enters a region of relatively constant stress with significant deformation or strain. This constant or plateau region is known as the loading stress, since it represents the behavior of the material as it encounters continuous increasing strain. It is in this plateau region CD that the transformation from austenite to martensite occurs.

At point D the transformation to the martensitic phase due to the application of stress to the specimen is substantially complete. Beyond point D the martensitic phase begins to deform, elastically at first, but, beyond point E, the deformation is plastic or permanent.

When the stress applied to the superelastic metal is removed, the material behavior follows the curve from point E to point F. Within the E to F region, the martensite recovers its original shape, provided that there was no permanent deformation to the martensitic structure. At point F in the recovery process, the metal begins to transform from the stress-induced, unstable, martensitic phase back to the more stable austenitic phase.

In the region from point G to point H, which is also an essentially constant or plateau stress region, the phase transformation from martensite back to austenite takes place. This constant or plateau region GH is known as the unloading stress. The line from point I to the starting point A represents the elastic recovery of the metal to its original shape.

Binary nickel-titanium alloys that exhibit superelasticity have an unusual stress-strain relationship as just described and as plotted in the curve of FIG. 6. As emphasized above, the superelastic curve is characterized by regions of nearly constant stress upon loading, identified above as loading plateau stress CD and unloading plateau stress GH. Naturally, the loading plateau stress CD is always larger than the unloading plateau stress GH. The loading plateau stress represents the period during which martensite is being stress-induced in favor of the original austenitic crystalline structure. As the load is removed, the stress-induced martensite transforms back into austenite along the unloading plateau stress part of the curve. The difference in stress between the stress at loading CD and unloading stress GH defines the hysteresis of the system. This is identified as Δy of the curve in FIG. 6.

The present invention seeks to minimize the hysteresis of the superelastic material used to fabricate stent 10. Stent 10 is designed to perform various mechanical functions within a lumen, all of which are based upon the lower unloading plateau stress GH. Unloading plateau stress GH represents the behavior of the nitinol material when the stent is deployed.

On the other hand, the higher loading plateau stress CD establishes the mechanical resistance stent 10 exerts against the delivery system, and specifically delivery sheath 16. It represents the stress exerted by stent 10 when it is loaded into sheath 16. The greater the difference between the two plateaus CD and GH is (the hysteresis), the stronger the delivery system must be to accommodate any given level of stent performance. A stronger delivery system must necessarily be larger and bulkier, with a thicker delivery sheath 16.

Conversely, reducing the difference or Δy between the two plateaus CD and GH results in smaller hysteresis. The smaller the hysteresis is, the smaller and lower profile the delivery system has to be to accommodate any given level of stent performance. Furthermore, the present invention delivery system can be smaller and constructed to a smaller profile due to the lower loading plateau stress CD, while maintaining a high hoop strength of the deployed, expanded stent represented by plateau stress GH.

In accordance with the present invention, stent 10 requires only a delivery system having a small delivery profile P as illustrated in the cross-sectional views of FIGS. 1 and 2. Furthermore, the wall thickness 34, 36 can be reduced as compared to a comparable performance stent not employing the present invention. Such a compact delivery system permits the physician better access and flexibility to reach tortuous arteries and vessels.

In sum, the present invention offers the potential to reduce overall delivery profile defined by loading stress CD for any given level of stent mechanical performance defined by unloading stress GH. In the present invention, this is accomplished by realizing the properties of superelastic nitinol, preferably in addition with a ternary element, as described in greater detail below.

The superelastic alloy of the present invention is preferably formed from a composition consisting essentially of about 30 to about 52 percent titanium and the balance nickel and up to 10 percent of one or more additional ternary alloying elements. Such ternary alloying elements may be selected from the group consisting of palladium, chromium, iron, cobalt, vanadium, manganese, boron, copper, aluminum, tungsten, or zirconium. In particular, the ternary element may optionally be up to 3 percent each of iron, cobalt, platinum, palladium, and chromium, and up to about 10 percent copper and vanadium. As used herein, all references to percent composition are atomic percent unless otherwise noted.

In another preferred embodiment, a NiTi stent with SME (shape memory effect) is heat-treated at approximately 500 degrees C. The stent is mechanically deformed into a first, smaller diameter for mounting on a catheter delivery system, such as the delivery system of FIG. 2, that includes expandable balloon 20 and balloon inflation lumen 22. After the stent has been expanded by the balloon and deployed against arterial wall 29 of artery 28, 45 degrees C. heat is applied causing the stent to return to its fully expanded larger diameter and be in contact with the arterial wall of the artery. The application of 45 degrees C. of heat is compatible with most applications in the human body, but it is not to be limited to this temperature as higher or lower temperatures are contemplated without departing from the invention. The 45 degrees C. temperature can be achieved in a conventional manner well known in the art such as by warm saline injected into the delivery catheter and balloon.

The shape memory characteristics allow the devices to be deformed to facilitate their insertion into a body lumen or cavity and then to be heated within the body so that the device returns to its original shape. Again, alloys having shape memory characteristics generally have at least two phases: a martensitic phase, which has a relatively low tensile strength and which is stable at relatively low temperatures, and an austenitic phase, which has a relatively high tensile strength and which is stable at temperatures higher than the martensitic phase.

Shape memory characteristics are imparted to the alloy by heating the metal to a temperature above which the transformation from the martensitic phase to the austenitic phase is complete; i.e., a temperature above which the austenitic phase is stable. The shape of the metal during this heat treatment is the shape “remembered.” The heat-treated metal is cooled to a temperature at which the martensitic phase is stable, causing the austenitic phase to transform to the martensitic phase. The metal in the martensitic phase is then plastically deformed, e.g., to facilitate the entry thereof into a patient's body. Subsequent heating of the deformed martensitic phase to a temperature above the martensite to austenite transformation temperature causes the deformed martensitic phase to transform to the austenitic phase. During this phase transformation the metal reverts back to its original shape.

The recovery or transition temperature may be altered by making minor variations in the composition of the metal and in processing the material. In developing the correct composition, biological temperature compatibility must be determined in order to select the correct transition temperature. In other words, when the stent is heated, it must not be so hot that it is incompatible with the surrounding body tissue. Other shape memory materials may also be utilized, such as, but not limited to, irradiated memory polymers such as autocrosslinkable high density polyethylene (HDPEX).

Shape memory alloys are known in the art and are discussed in, for example, “Shape Memory Alloys,” Scientific American, Vol. 281, pp. 74-82 (November 1979), incorporated herein by reference.

Shape memory alloys undergo a transition between an austenitic state and a martensitic state at certain temperatures. When they are deformed while in the martensitic state they will retain this deformation as long as they are retained in this state, but will revert to their original configuration when they are heated to a transition temperature, at which time they transform to their austenitic state. The temperatures at which these transitions occur are affected by the nature of the alloy and the condition of the material. Nickel-titanium-based alloys (NiTi), wherein the transition temperature is slightly lower than body temperature, are preferred for the present invention. It is desirable to have the transition temperature set at just below body temperature to insure a rapid transition from the martensitic state to the austenitic state when the stent is implanted in a body lumen.

Turning again to FIG. 3, stent 10 is formed from a shape memory alloy, such as NiTi discussed above. After stent 10 is inserted into artery 28 or other vessel, expandable balloon 20 is inflated via balloon inflation lumen 22 by conventional means such that the stent is expanded radially outwardly. The stent then immediately expands due to contact with the higher temperature within artery 28 as described for devices made from shape memory alloys. Again, if an external force is then applied to the artery, stent 10 temporarily at least partially collapses. But stent 10 then quickly regains its former expanded shape due to its shape memory qualities. Thus, the crush-resistant stent, having shape memory characteristics, is implanted in a vessel, thereby maintaining the patency of a vessel while minimizing both the risk of permanent vessel collapse and the risk of dislodgment of the stent from the implant site if the stent is temporarily deformed due to external forces.

While the present invention has been illustrated and described herein in terms of a superelastic stent and delivery system wherein the stent employs a ternary element to minimize the hysteresis defined by the difference in the loading plateau stress and the unloading plateau stress of the superelastic material, it is apparent to those skilled in the art that the present invention can be used in other instances. Other modifications and improvements may be made without departing from the scope of the present invention.

What is claimed is:

1. A stent and a delivery system for implanting the stent in a body lumen, comprising:
a cylindrically-shaped stent including a superelastic alloy, wherein the alloy includes a ternary element, and wherein the alloy further includes a substantially small stress hysteresis; and
a delivery system including a sheath having a distal end and a proximal end, wherein the stent is disposed inside the sheath at the distal end, and wherein the delivery system has a small profile.

2. The stent and delivery system of claim 1, wherein the superelastic alloy includes a nickel-titanium alloy.

3. The stent and delivery system of claim 1, wherein the ternary element is selected from the group of elements consisting of palladium, chromium, iron, cobalt, vanadium, manganese, boron, copper, aluminum, tungsten, or zirconium.

4. The stent and delivery system of claim 1, wherein the small stress hysteresis is defined by a curve plotted on right angle axes wherein a y-axis scale represents stress versus an x-axis scale that represents strain, and wherein a Δy of the curve is small.

5. The stent and delivery system of claim 1, wherein the small stress hysteresis represents minimal difference between a loading stress and an unloading stress of the alloy.

6. The stent and delivery system of claim 1, wherein the sheath includes a thin wall.

7. The stent and delivery system of claim 1, wherein the stent includes independently expandable cylindrical elements.

8. The stent and delivery system of claim 1, wherein the delivery system includes an inner member having a balloon at the distal end, the inner member having an inflation lumen therein in fluid communication with an interior of the balloon, and wherein the sheath is slidably disposed over at least a portion of the inner member.

9. The stent and delivery system of claim 1, wherein the stent includes a nested strut pattern.

10. A stent and a delivery system for implanting the stent in a body lumen, comprising:
a self-expanding, cylindrically-shaped stent including a nickel-titanium alloy, wherein the nickel-titanium alloy includes a ternary element, and wherein the alloy further includes a substantially small stress hysteresis;
a delivery system including an inner member having a distal end and a proximal end, wherein the stent is disposed at the distal end; and
the delivery system further including a sheath having a distal end and a proximal end, wherein at least the distal end of the sheath is slidably disposed over the stent, and wherein the delivery system has a small profile.

11. The stent and delivery system of claim 10, wherein the ternary element is selected from the group consisting of palladium, chromium, iron, cobalt, vanadium, manganese, boron, copper, aluminum, tungsten, or zirconium.

12. The stent and delivery system of claim 10, wherein the small stress hysteresis is defined by a curve plotted on right angle axes wherein a y-axis scale represents stress versus an x-axis scale that represents strain, and wherein a Δy of the curve is small.

13. The stent and delivery system of claim 10, wherein the small stress hysteresis represents minimal difference between a loading stress and an unloading stress of the alloy.

14. The stent and delivery system of claim 10, wherein the inner member includes a balloon at a distal end and an inflation lumen therein in fluid communication with an interior of the balloon.

15. The stent and delivery system of claim 10, wherein the sheath includes a thin wall.

16. The stent and delivery system of claim 10, wherein the alloy includes not more than 10 atomic percent of the ternary element.

17. A method for implanting a stent in a body lumen, comprising the steps of:
providing a self-expanding, cylindrically-shaped stent including a nickel-titanium alloy, wherein the nickel-titanium alloy includes a ternary element, and wherein the alloy further includes a substantially small stress hysteresis;
providing a delivery system including an inner member having a distal end and a proximal end, wherein the delivery system has a small profile;
disposing the stent at the distal end of the delivery system;
providing a sheath as part of the delivery system, the sheath having a distal end and a proximal end; and
slidably disposing the sheath over the stent.

18. The method for implanting a stent in a body lumen of claim 17, wherein the step of providing a self-expanding, cylindrically-shaped stent includes selecting the ternary element from the group consisting of palladium, chromium, iron, cobalt, vanadium, manganese, boron, copper, aluminum, tungsten, or zirconium.

19. The method for implanting a stent in a body lumen of claim 17, wherein the step of providing a self-expanding, cylindrically-shaped stent includes defining the small stress hysteresis by a curve plotted on right angle axes wherein a y-axis scale represents stress versus an x-axis scale that represents strain, and wherein a Δy of the curve is small.

20. The method for implanting a stent in a body lumen of claim 17, wherein the step of providing a delivery system includes providing a balloon at the distal end of the delivery system and providing a lumen within the delivery system in fluid communication with the balloon.
